(19) United States
(12) Patent Application Publication
(10) Pub. No.: US 2025/0114495 A1
(43) Pub. Date: Apr. 10, 2025

(54) AIR PURIFICATION SYSTEM AND METHOD

(71) Applicant: Clean Air Zone Inc., Corona, NY (US)

(72) Inventors: Serge B. HOYDA, Great Neck, NY (US); Sravani GANDU, Forest Hills, NY (US); Nicolas MORENO, Corona, NY (US)

(73) Assignee: Clean Air Zone Inc., Corona, NY (US)

(21) Appl. No.: 18/656,997

(22) Filed: May 7, 2024

Related U.S. Application Data
(60) Provisional application No. 63/589,215, filed on Oct. 10, 2023.

(51) Int. Cl. A61L 9/14; B01D 53/34; B01D 53/84

(52) U.S. Cl. CPC A61L 9/145; B01D 53/346; B01D 53/84; A61L 2209/111; A61L 2209/134; A61L 2209/14; B01D 2258/06

(57) ABSTRACT

At least one embodiment relates to an air purification system comprising a housing, at least one fan, a pump and at least one controller coupled to said housing wherein the controller is configured to control the pump and the fan. There is at least one air intake formed in the housing, at least one biological solution comprising a biological agent and water, disposed in the housing. There is at least one spray nozzle disposed in said housing. The pump is configured to pump the biological solution through the spray nozzle to spray the biological solution from said spray nozzle onto a screen. The screen is positioned in the housing, in a substantially vertical orientation and is configured to interact with air flowing through the housing.

### CROSS REFERENCE TO RELATED APPLICATIONS

This application is a non-provisional application and hereby claims priority from provisional application Ser. No. 63/589,215 filed on Oct. 10, 2023, the disclosure of which is hereby incorporated by reference in its entirety.

### BACKGROUND OF THE INVENTION

One embodiment of the invention relates to an air purification system and method. At least one embodiment relates to an air purification system based on an environmentally safe microbial biologic al solution. This microbial biological solution when combined with water is called biological agent.

There is an air purification system configured to create an interaction between a biological agent and air flow, wherein the interaction between the biological agent and the airflow cleanses the air. Previously air purification systems would be hindered by lack of interaction between the air flowing through the air purifier and the interaction with the biological agent. Therefore, there is a need to have a plurality of different interaction surfaces configured to receive biological agent so that there is increased interaction with air flow through an air purification system.

There is a need for an air purification system which is configured to be small and compact and to treat air through purifying the air in a compact yet effective manner.

### SUMMARY

At least one embodiment relates to an air purification system comprising a housing, at least one fan, a pump and at least one controller coupled to said housing wherein the controller is configured to control the pump and the fan. There is at least one air intake formed in the housing, at least one biological solution comprising a biological agent and water, disposed in the housing. There is at least one spray nozzle disposed in said housing. The pump is configured to pump the biological solution through the spray nozzle to spray the biological solution from said spray nozzle onto a screen. The screen is positioned in the housing, in a substantially vertical orientation and is configured to interact with air flowing through the housing.

### BRIEF DESCRIPTION OF THE DRAWINGS

Other objects and features of the present invention will become apparent from the following detailed description considered in connection with the accompanying drawings which disclose at least one embodiment of the present invention. It should be understood, however, that the drawings are designed for the purpose of illustration only and not as a definition of the limits of the invention.

In the drawings, wherein similar reference characters denote similar elements throughout the several views:

FIG. 1A is a top front right perspective view of the air purification system;

FIG. 1B is a top view of the air purification system;

FIG. 2A is a front view of the air purification system;

FIG. 2B is a back view of the air purification system;

FIG. 3 is a front-top perspective exploded view of the air purification system;

FIG. 4 is a top transparent view of the air purification system;

FIG. 5 is another exploded view of the air purification system;

FIG. 6 is a side transparent view of the air purification system;

FIG. 7A is a side view of the air purification system with the cover or housing removed;

FIG. 7B is a top view of another embodiment showing the fan and the intake on the top surface of the housing;

FIG. 8 is a plan view of the air purification system disclosing the electronic components;

FIG. 9 is a block diagram of the network of components controlled or in communication with the controller; and

FIG. 10 is a flow chart for the process for purifying air.

FIG. 11A is first perspective view of another embodiment of the invention;

FIG. 11B is a side view of the embodiment of FIG. 11A;

FIG. 12A is another side view of the embodiment of FIG. 11A;

FIG. 12B is another perspective view of the embodiment of FIG. 11A;

FIG. 13A is a top view of the embodiment of FIG. 11A;

FIG. 13B is a side cross-sectional view of the embodiment of FIG. 11A;

FIG. 14 is a cut away view of the inner portion of the cylindrical housing;

FIG. 15 is a view of the electronic components of the device;

FIG. 16 is a flow chart for the process for using the embodiment of FIG. 11A;

FIG. 17 is another side view of another embodiment;

FIG. 18 is a top view of another embodiment;

FIG. 19 is a side view of another embodiment;

FIG. 20A is a top view of another embodiment;

FIG. 20B is a side view of another embodiment;

FIG. 21 is a top view of one embodiment of the air purification device;

FIG. 22 is a side view of the air purification device;

FIG. 23 is a block diagram layout of the air purification device;

FIG. 24 is a flow chart of the process for cleaning device.

FIG. 25A is a view of a first embodiment of a level sensor; and

FIG. 25B is a view of the second embodiment of the level sensor.

### DETAILED DESCRIPTION

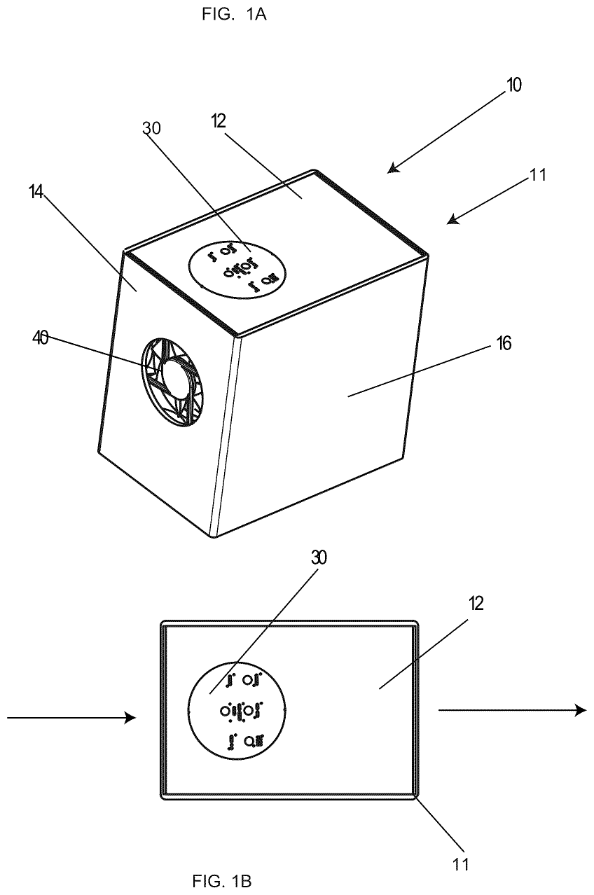

FIG. 1A is a front right top perspective view of the air purification system 10 which discloses a housing 11 having a top side 12, a front side 14, a right side 16 as well as a controller or control panel 30. There is also a fan 40 positioned on the front side 14. The fan 40 can be configured to provide positive pressure inside of the housing 11 or the fan 40 can be reversed so that it can create negative pressure inside of the housing. FIG. 1B is a top view which shows controller 30 positioned on top side 12 with the arrows showing the air flow through housing 11.

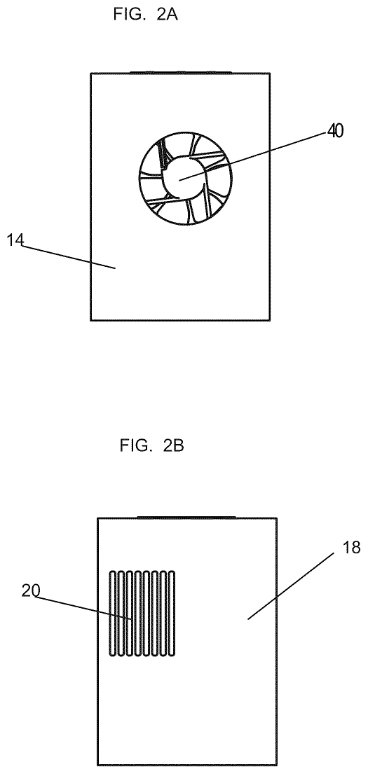

FIG. 2A shows a front view of the device showing front side 14 as well as fan 40. FIG. 2B shows a back side view showing back side 18 with louvers 20 positioned on the back side. The fan 40 on front side 14 and the louvers 20 on back side 18 allow for a passage of air or fluid flow through the housing 11 thereby allowing air to interact with biological solution inside of the housing 11. While louvers 20 are shown, screens or other suitable covers can also be used as well. The interaction between the biological solution and the air results in the impurities being electrostatically and biologically removed from the air to thereby purify the air.

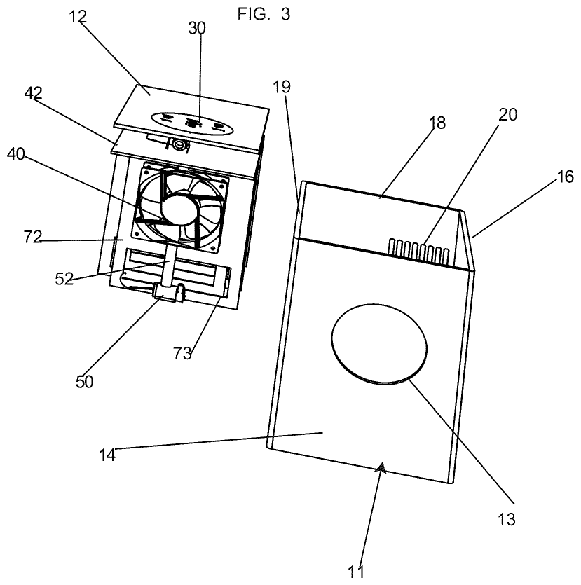

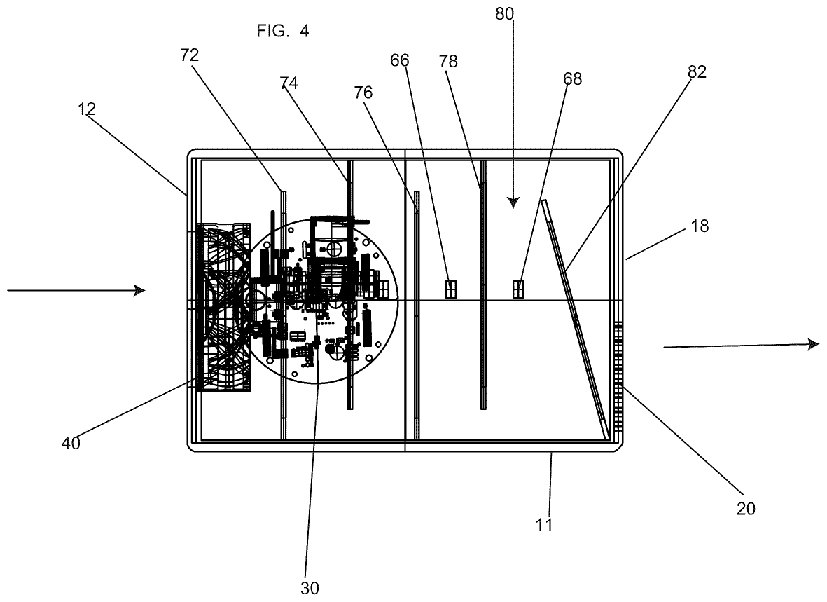

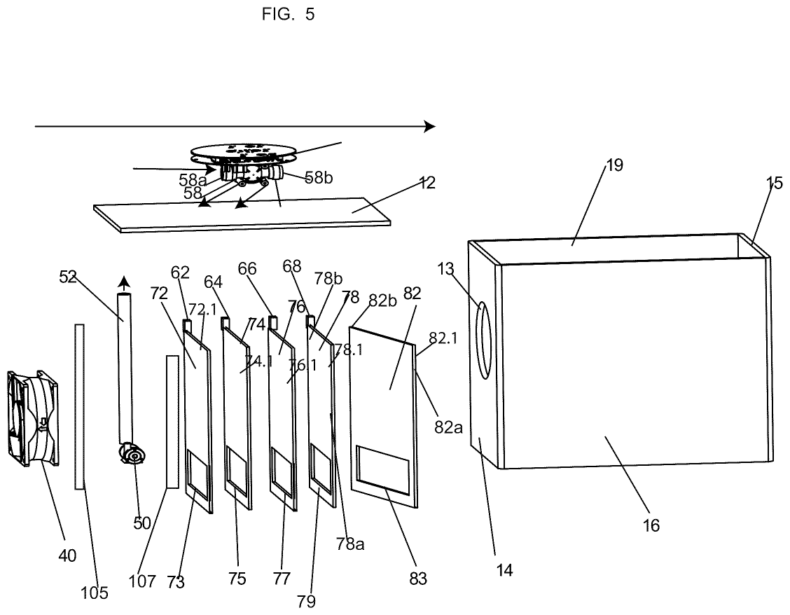

FIG. 3 is a perspective front top exploded view of the device with the cover or housing 11 being removed from the device. This view shows cover or housing 11 removed wherein housing 11 includes right side 16, front side 14, hole 13, left side 19 and back side 18 with louvers 20. The components shown outside of housing 11 in this view are top side 12, controller 30, second level 42, fan 40, pump 50 as well as first divider 72 with opening 73 in first divider 72. Pump 50 is for pumping fluid such as the biological solution into the nozzles such as nozzles 62, 64, 66, and 68 (See FIGS. 4 and 5).

FIG. 4 is a top view of device 10 which shows housing 11 having a front side 14, dividers 72, 74, 76, 78, and 82. Inside of housing 11 is biological solution 80 which rests on the bottom of the housing 11 and which moves freely between dividers 72, 74, 76, 78, and 82. There are a plurality of nozzles disposed inside of housing 11, wherein the nozzles shown include nozzles 62, 64, 66 and 68 (See also FIG. 5). In addition, controller 30 is shown positioned above fan 40. The arrow positioned to the left of fan 40 is shown as the air intake wherein the arrow positioned to the right of the louvers 20 shows the air outflow from the housing 11. Alternatively, as disclosed above, the air flow can be reversed so that the air flow is in the opposite direction wherein fan 40 creates a negative pressure inside of the housing 11.

FIG. 5 is an exploded side view of the device wherein there is shown housing 11 having a front side 14, a hole 13 for receiving fan 40, a right side 16 a back side 18, a left side 19 a top 12, controller 30, a second pump 90 coupled to controller 30, another pump 50 a channel 52 coupled to pump 50 as well as screens or dividers 72, 74, 76, 78, and 82. Screen 72 has opening 73, screen 74 has opening 75, screen 76 has opening 77 screen 78 has opening 79, and screen 82 has opening 83. These openings 73, 75, 77, 79 and 83 allow for fluid in the form of biological solution 80 to flow from the front to the back and also from the back to the front of the housing. Pump 50 is configured to pump the biological solution 80 up channel 52 to a top region wherein it is taken via a conduit to nozzles 62, 64, 66 and 68. Nozzles 62, 64, 66, and 68 are configured to spray biological solution onto primarily a top half of the adjacent screens 72, 74, 76, 78, and 82. These top halves are shown as top half 72.1, 74.1, 76.1, 78.1, and 82.1. The nozzles 62, 64, 66, and 68 are configured to spray the biological solution in a 360 degree or omnidirectional manner so that the biological solution is sprayed on both sides of the screens 72, 74. 76, 78, and 82. In addition in alternative embodiments, additional screens such as additional screen 84 can be added inside of housing 11, wherein housing 11 can be elongated further if necessary. An additional nozzle such as nozzle 69 can be added as well inside of the housing to spray on additional screen 84. Additional screen 84 has a top portion 84.1 a front end 84a, a back end 84b as well as an opening 85 for allowing biological solution to flow therethrough. In addition, an optional additional mixing pump 51 can be placed inside of housing 11 to pump and mix the biological solution around so that it doesn't fully coagulate as sediment.

These screens are orientated substantially vertically inside of housing 11 when the device is positioned in an upright operating position. When the biological solution is sprayed on these screens it is sprayed on a top portion of these screens such as in regions 72.1, 74.1, 76.1, 78.1, 82.1 and 84.1 creating a mist. The top portions of the screens receive the biological solution so that the solution can then drip down the sides of the screens and into the remainder of the solution inside of the housing.

The air interacting with the biological solution in the form of a mist as well as the air interacting with the biological solution moving down the sides of the dividers or screens causes an interaction between the solution and the air so that there is both a chemical and electrostatic interaction resulting in the impurities being removed from the air. Because the biological solution is sprayed in the form of a mist it allows many particles of biological solution to interact with the air drawing the particles or impurities out of the air. In addition, the positioning of the dividers or screens inside of the housing is such that the air flow is designed to interact with the screens and the biological solution flowing down the screens to further increase interaction between the air and the biological solution. In particular at least the last screen 82 (or screen 84) is angled in such a way that the first end 82a of the screen is farther away from the first end 78a of screen 78. However, the second end 82b of screen 82 is closer in distance to the second end 78b of screen 78. This angling of the screens or at least screen 82 allows for increased air pressure and thereby increased interaction between the air and the biological solution. In addition, there is also shown two different level sensors 105 and 107. These level sensors are described in greater detail in U.S. patent application Ser. No. 17/852,341 filed on Jun. 28, 2022, the disclosure of which is hereby incorporated herein by reference in its entirety. Any one or both of the two-level sensors can be used inside of the housing with the second level sensor being a safety level sensor to make sure that the housing does not overfill with water or biological solution.

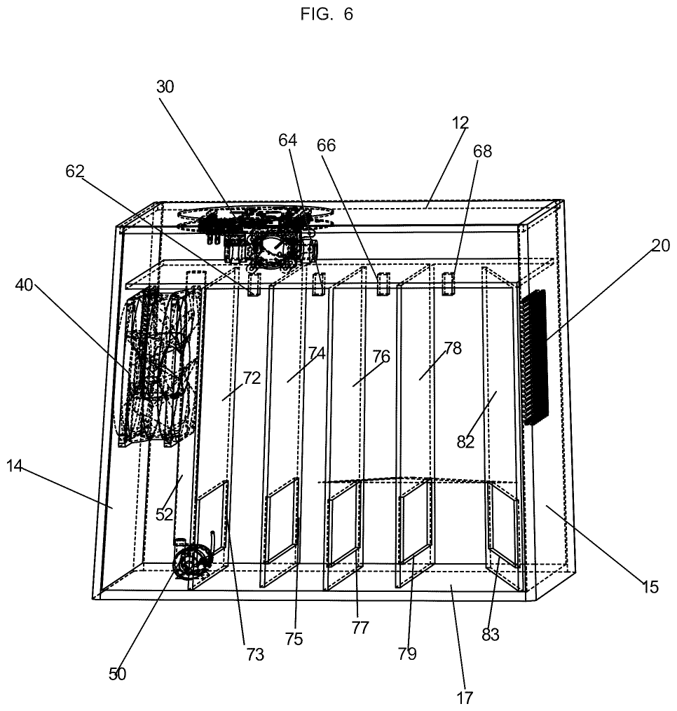

FIG. 6 is a side transparent view of the air purification system which shows front side 14, fan 40, controller 30, pump 50, channel 52, nozzles 62, 64, 66, 68, screen 72 having opening 73, screen 74, having opening 75, screen 76 having opening 77, screen 78, having opening 79 and screen 82 having opening 83. Backside 18 shows louvers 20 as well. As described above the nozzles spray biological solution on the respective screens in an omnidirectional manner. For example, nozzle 62 sprays biological solution on screens 72 and 74, nozzle 64 sprays biological solution on screens 74 and 76, nozzle 66 sprays biological solution on screens 76 and 78, while nozzle 68 sprays biological solution on screens 78, and 82. As shown both sides of screens 74, 76 and 78 have biological solution sprayed on them such that for example screen 74 has solution sprayed on a first side via nozzle 62, and on a second side via nozzle 64. Screen 76 has a solution sprayed on a first side via nozzle 64 and on a second side via nozzle 66. Screen 78 has a solution sprayed on a first side via nozzle 66 and on a second side via nozzle 78.

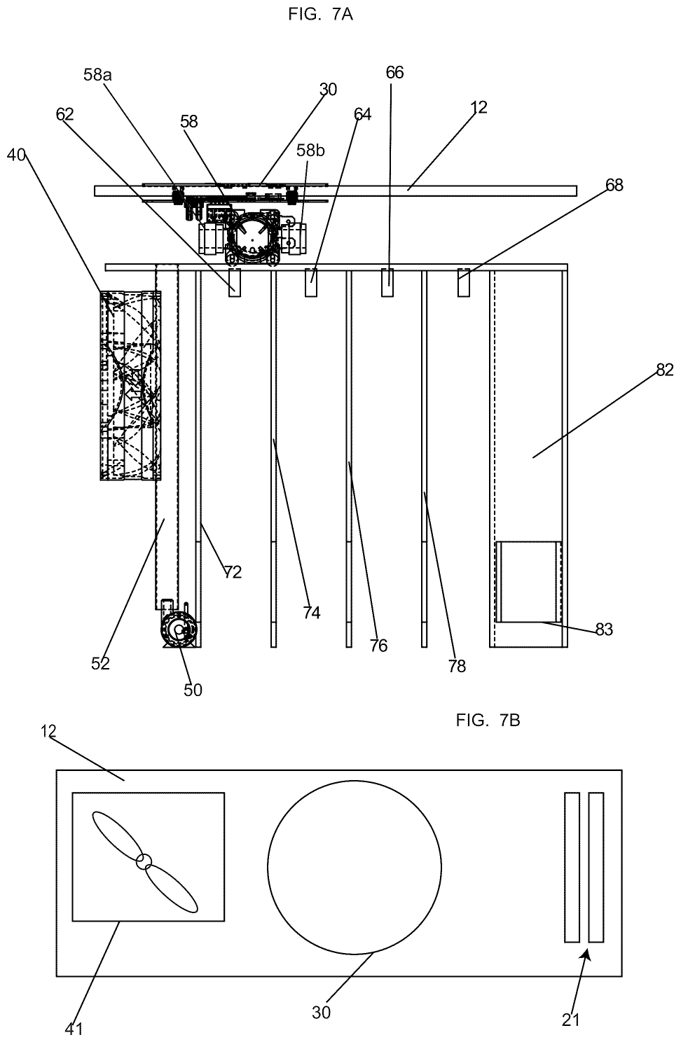

FIG. 7A shows a side view with the housing 11 removed. This view shows fan 40, controller 30, solenoid valve 58, nozzles 62, 64, 66, and 68 which sprays biological solution on screens 72, 74, 76, 78 as described above, pump 50 is shown with column or channel 52 wherein pump 50 can also be a circulating pump for circulating the material or solution throughout the housing. Solenoid valve 58 receives water inflow from an external water source to allow additional water to flow inside of housing 11. Pump 50 pumps biological solution through channel 52 and provides a pressurized fluid flow to nozzles 62, 64, 66, and 68 so that the spray from the nozzles forms a particularized mist for interacting with the impure air. The mist then flows due to the air pressure into the walls of the screens 72, 74, 76, 78, and then flows down the walls of the screens into the pool of biological solution on the bottom of housing 11. In this way, because the mist is trapped on the walls, it doesn't flow out from the housing thereby causing the loss of the biological solution from the inside of the housing 11. Otherwise, the mist would flow from the front of the housing to the back of the housing caused by the pushing of the air inside of the housing from the fan 40, without the screens some mist would then flow outside of the housing through louvers 20. In at least one embodiment, the louvers are orientated vertically. In at least one embodiment the louvers are orientated substantially horizontally and pointing up so that any remaining mist is trapped by the louvers and fluid flows back into the housing thereby trapping the fluid inside of the housing.

In at least one embodiment, valve 58 is in the form of an inlet valve for adding more external fluid such as water into the system wherein section 58a is a water inlet from an external water source and section 58b with a solenoid disposed in between controlling a water outflow for allowing water to flow through the pump or valve into the housing.

FIG. 7B is a top view of another embodiment of the device which shows fan 41 positioned on a top surface 12 along with an air intake 21 or outflow depending on the flow rate of the fan 41 whether it creates a positive pressure or a negative inside of the housing. With the fan 41 and the air intake or outflow 21 being positioned on top of the housing this allows for the moist air to drop any of the droplets back into the housing section 11 before the air leaves the housing 11. Controller 31 is essentially the same as control panel 30 however this control panel is rectangular and is positioned on the top surface 12.

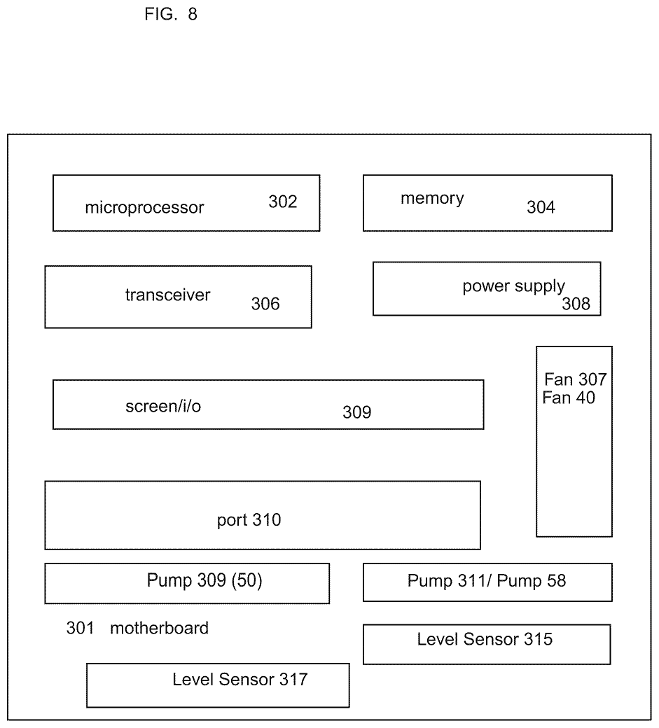

FIG. 8 is a schematic block diagram of the electronic components for controller 30. For example, there is a motherboard 301, disposed on the motherboard is microprocessor 302, a memory 304, a transceiver 306, a power supply 308, a screen 309 which is a touch screen which allows for the selective input of instructions such as increasing the fan rate or pump rate of the biological solution or increasing the amount of water input into the system. The screen also includes an on/off button to turn on or off the machine as well. There is a fan controller 307 for controlling the fan based upon instructions sent from microprocessor 302. There is also a pump controller 309 for controlling pump 50 as well as a valve controller 311 for controlling valve 58. As indicated above, valve 58 can be used to selectively control the flow rate of fluid through the selective nozzles or it can be used to selectively control the inflow of new water into the system. There are also level sensor ports 315 and 317 for communicating with selective level sensors disposed inside of the housing. These level sensors are described in greater detail in U.S. patent application Ser. No. 17/852,341 filed on Jun. 28, 2022, the disclosure of which is hereby incorporated herein by reference in its entirety.

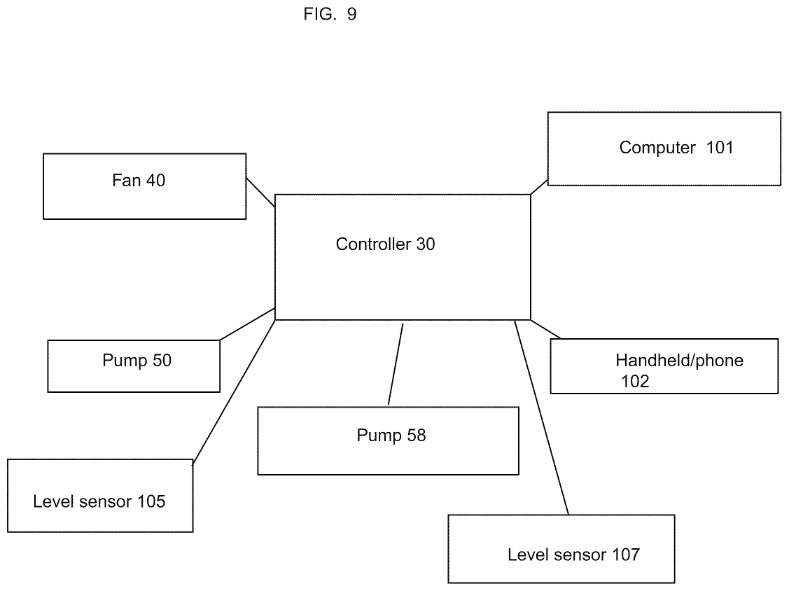

FIG. 9 is a network diagram of the system which includes the controller 30 shown in FIG. 8, as well as the peripheral devices such as the fan 40, the pump 50, the valve 58, the level sensor 105, the level sensor 107 as well as computer 101 and/or a portable handheld device such as a smartphone or tablet. Either the computer 101 or the handheld or portable device 102 can be used to remotely control controller 30 through transceiver 306 (See FIG. 8) wherein transceiver 306 is a wireless transceiver such as Cellular, WIFI, Bluetooth or nearfield or any other suitable wireless protocol. Thus, the device can be controlled to control the fan speed RPM, the fluid flow through the pump 50 or add water through solenoid valve 58, or to shut water input down via sensors of the level sensors 105 and 107, to regulate the amount of fluid, the amount of biological solution, the fan speed and flow rate to optimize the air purification benefits of the device.

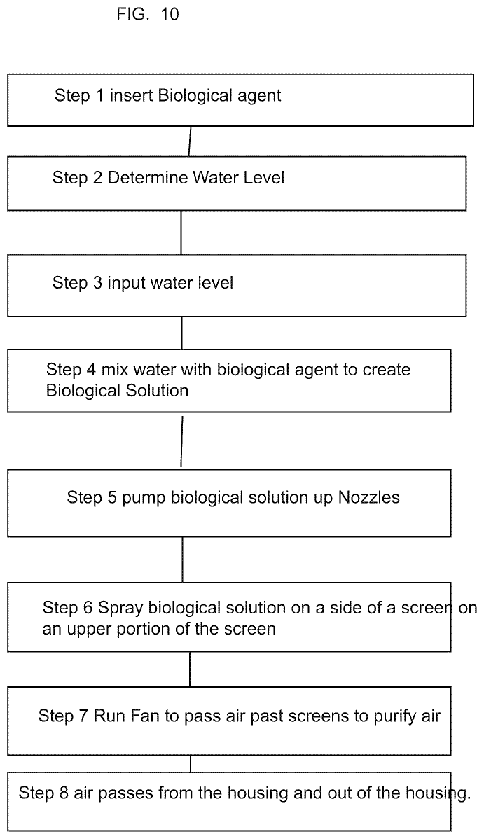

FIG. 10 is a process for cleansing air using the device disclosed above. For example, the process starts in step 1 wherein a user could insert the biological agent inside of housing 11. Next, in step 2 the system using the water level sensors 105 and 107 can determine the water. Next, in step 3 the water input valve 58 can allow water to flow into the housing to raise the water level to a pre-determined level inside of the housing. Next, in step 4 the system can include mixing the water with the biological agent to create the biological solution. This can occur via the pump 50 circulating the water inside of housing 11. Next, in step 5 the process can include pumping the biological solution up to nozzles 62, 64, 66, and 68 using pump 50. In at least one embodiment the valve 58 can be formed as an auxiliary pump in communication with the water feed from pump 50 to press the water through nozzles 62, 64, 66 and 68 to spray onto the screens 72, 74, 76, 78, and 82. In step 6 the process includes spraying the solution on an upper portion of the screens so that this solution drips down the side of the screens. Next as the solution drips down the side of the screens the fan in step 7 is moving to push the air past the screens 72, 74, 76, 78 and 82 so that the air interacts with the solution to cleanse the air. The air then passes past the louvers 20 and out of the housing 11 in step 8.

The housing forms a container for the biological agent so that it can be pumped to different locations for eventual interaction with air. The biological agent is configured to be mixed with air so that it cleanses the air of biological material and therefore provides cleaner air from the air purification system.

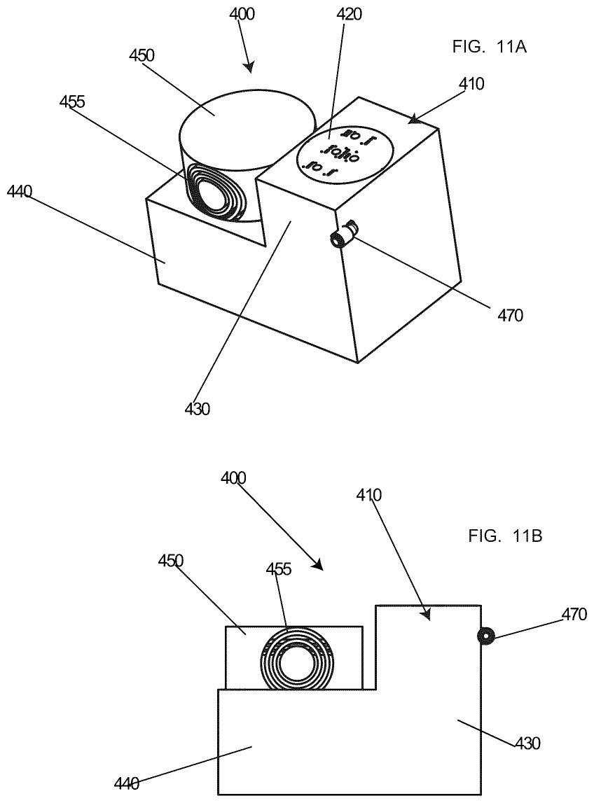

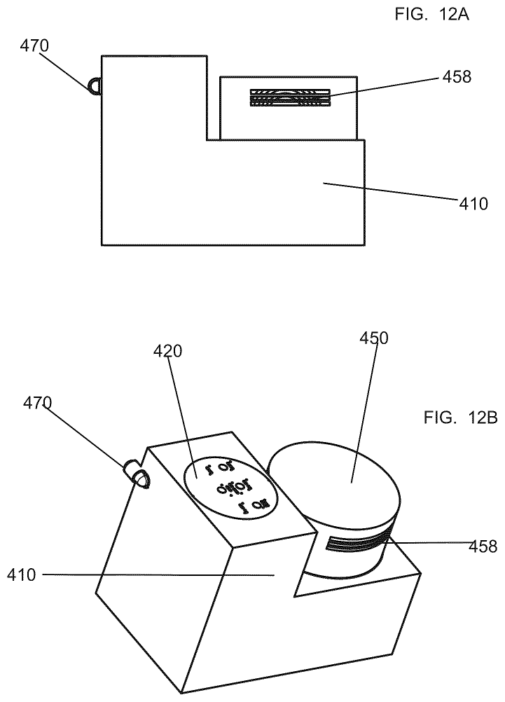

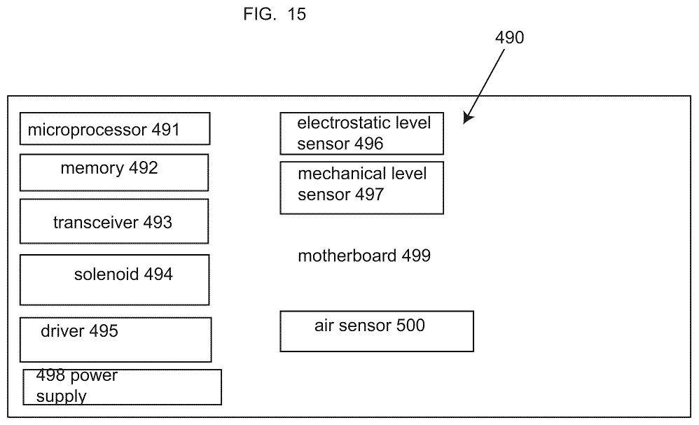

FIGS. 11A, 11B, 12A, 12B, show another embodiment, which is an air purification system 400 which has a housing 410. Housing 410 has three separate housings, a first housing portion 430, a second housing portion 440 and a third housing portion 450. Third housing portion 450 is a cylindrical housing portion which has an air inlet 455 and an air outlet 458. There is a controller 420 which has a set of controls for controlling the components inside of housing 410. There is also a fluid inlet 470 which is controlled by a solenoid (See FIG. 15) which allows water to flow into housing 410.

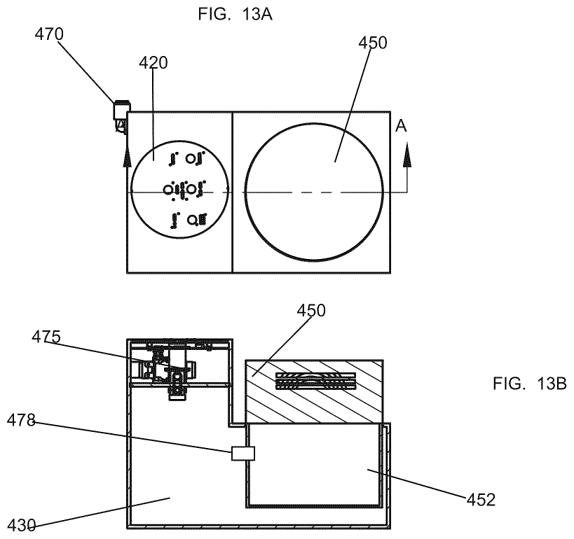

FIG. 13A shows the top view of the device which shows the third housing portion 450 as a cylindrically shaped housing, a top view of the controller 420 as well as the fluid inlet 470.

FIG. 13B shows a side cross-sectional view of device 400 which shows housing 410 having a first housing section 430, a second housing section 440 and a third housing section 450 having a chamber 452. Connecting chamber 452 with the housing section 430 is a fluid passage 478 which allows for fluid flow such as water or biological agent to flow between chamber 452 and housing section 430.

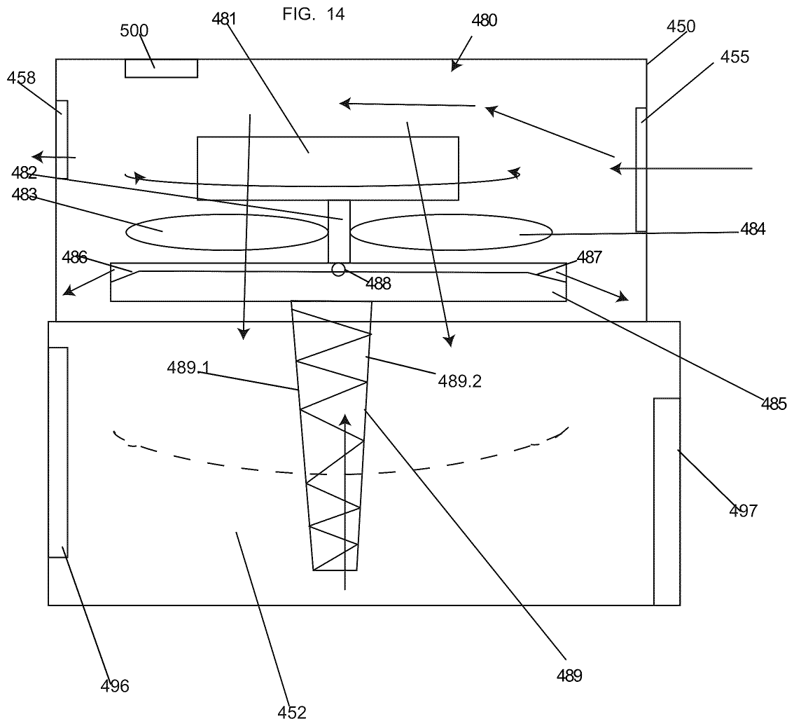

FIG. 14 shows a cross sectional view of the toroidal device 480 which is configured to generate air flow into the housing as well as generate interaction between the air flow and the biological agent. For example, there is shown an electrical motor or driver 481, which drives a drive shaft 482. Coupled to drive shaft 482 are blades 483, and 484. Drive shaft 482 is also coupled to turntable 485. Turntable 485 has a plurality of spouts 486, 487, and 488 dispersed around the turntable. Turntable 485 is substantially circular in shape and has different spouts extending out from it to allow water to flow out from turntable 485 when the turntable 485 is rotating. There is also a fluid inlet 489, which is configured to draw water up to turntable 485 which then allows this water to flow up from housing portion 440, through inlet 489 onto turntable 485 and then out against the side walls of housing 450 and then down to housing 440. Fluid inlet 489 is frusto-conical in shape and has expanding side walls up from a bottom section which is submerged in fluid such as biological solution. The expanding side walls (walls that are spaced farther apart from each other as the water travels up the fluid inlet, help to create a waterspout effect thereby drawing the water up the fluid inlet as the fluid inlet rotates within the biological solution. Thus, as the driver 481 which is an electrical motor is energized it rotates drive shaft 482, thereby driving blades 483, 484, as well as turntable 485 and fluid inlet 489. With this rotation, the blades create a driving force to force air through housing 450, while driving water up fluid inlet 489. When water and/or biological agent is drawn up inlet 489 it flows into turntable 485 and out of spouts 486, 487 and 488. This biological agent then flows centrifugally out interacting with the air flowing across the housing 450 thereby cleansing the air in the housing. The fan which comprises shaft 482, and blades 483, and 484 creates a negative pressure inside of housing 450 and draws air inward and downward into the biological solution allowing the biological solution to interact with the air so that the air can be purified before leaving the housing 450. The air after the interaction with the biological solution then leaves the housing via second opening 458. In addition, disposed inside of the housing is an electrostatic level sensor 496 as well as a mechanical level sensor 497 to check for the fluid levels inside of the housing. Furthermore, there is an air purification sensor 500 disposed inside of the housing as well to read the air quality level.

This system then creates a simplified air purification system which is configured to purify the air inside of the housing with one motor to drive both the air flow in and out of the housing as well as the flow of the biological solution up the fluid inlet and to spread the biological solution to increase the interaction between the biological solution and the air.

FIG. 15 is a view of the electronic components 490 of the device 400. These electronic components 400 include a motherboard 499, which is configured to house the different components. For example, there is a power supply 498, which powers the board to power the electronic components. There is also a microprocessor 491, which is configured to control the components such as the solenoid 494, the electric motor or driver 495 as well as the memory 492, and the transceiver 493. There are also two different level sensors including an electrostatic level sensor 496 and a mechanical level sensor 497. Both level sensors are configured to read the level of fluid such as biological solution that is present inside of housing 410. Thus, when microprocessor 491 reads the air quality level via air quality sensor 500, it can either increase or decrease the energy levels sent to motor or driver 495.

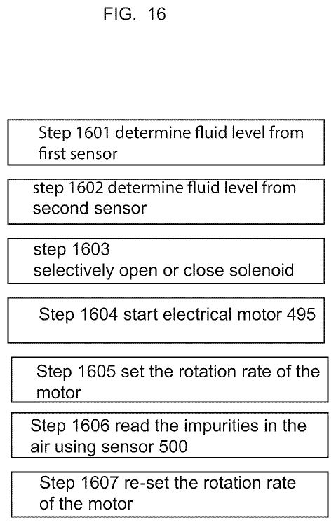

FIG. 16 is a view of the flow chart for using the device of FIG. 16. For example, there is a first step 1601 which includes determining a fluid level from a first sensor such as any one of the hydrostatic level sensor 496 or the mechanical level sensor 497. Next in step 1602 the system can determine the fluid level from the second sensor which can be any one of the hydrostatic sensor 496 or the mechanical level sensor 497 if the other sensor is used in the first step. Next, step 1603 if the system, particularly the microprocessor 491 can selectively determine whether to open or close the solenoid 494 to selectively allow water or fluid to flow into the housing. Next, in step 1604 the system, particularly microprocessor 491 can start the electrical motor 495 to start the fan and start the turning of the turntable as well as the fluid inlet 489. Next, in step 1605, the system such as the microprocessor can selectively set the speed of the rotation of the shaft 482 by setting the charge level or energy level being sent to motor 481. Next, in step 1606 the system can determine the amount of impurities in the air by using sensor 500. Once the system including microprocessor 491 determines the level of the impurities it next determines and re-sets the rates of rotation in step 1607 based upon the level of impurities in the air. Therefore, if there are a greater number of impurities detected by sensor 500, the rotation rate would increase thereby driving more air and water to interact with each other, thereby driving more purification of the air.

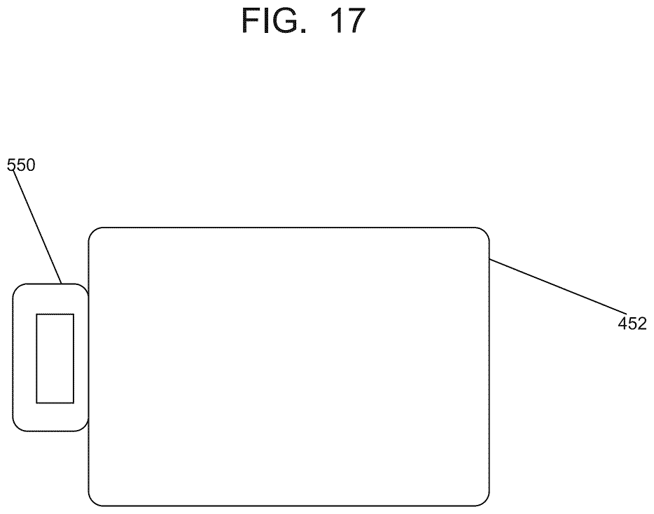

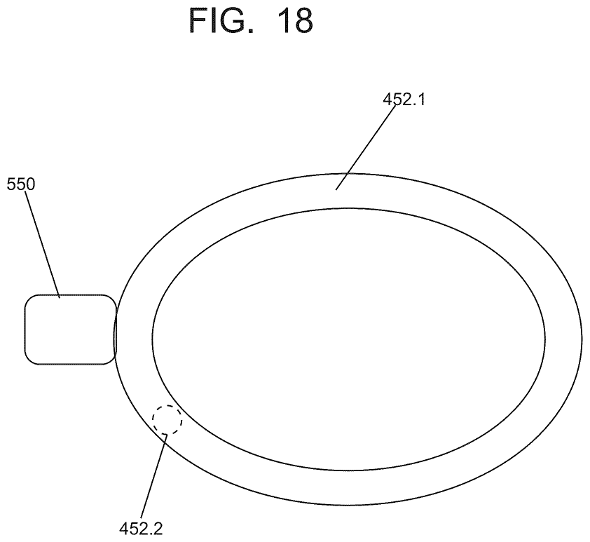

FIG. 17 is a side view of another embodiment which includes an enclosure or chamber 452 which has an associated handle 550 coupled thereto. FIG. 18 shows a top view of chamber 452 which has handle 550 as well as an optional opening 452.2 for receiving biological solution.

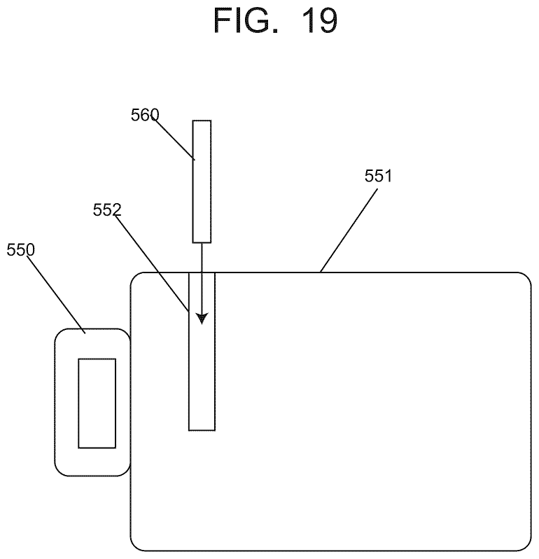

FIG. 19 is another view of another embodiment wherein there is shown a new chamber 551, which is coupled to handle 550. Coupled to chamber 551 is slot 552 for receiving a cartridge 560 to be inserted therein to the chamber. When the cartridge 560 is inserted into the chamber it releases biological solution into the chamber.

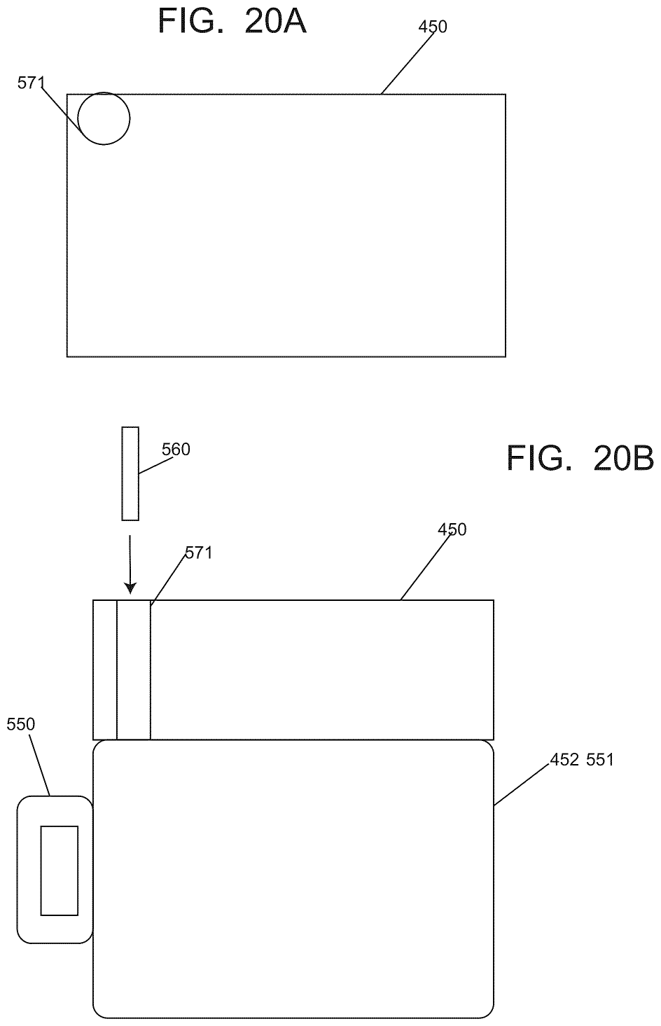

FIG. 20A is another view of another embodiment which shows a top view of a housing 450 having a slot 571 for receiving a cartridge such as cartridge 560 shown in FIG. 19. This cartridge would then extend through housing 570 and on towards chamber 452 or 551.

FIG. 20B is a side view of the device with housing 450 above chamber 452, 551. There is handle 550 coupled to this chamber. Slot 571 is formed inside of housing 450 which is configured to receive a cartridge 560. The cartridge contains biological solution which can be injected into chamber 452, 551 when cartridge 560 is inserted into the slot. The biological solution then flows into chamber 452, 551 so that it can them mix with water or a combination of water and biological solution.

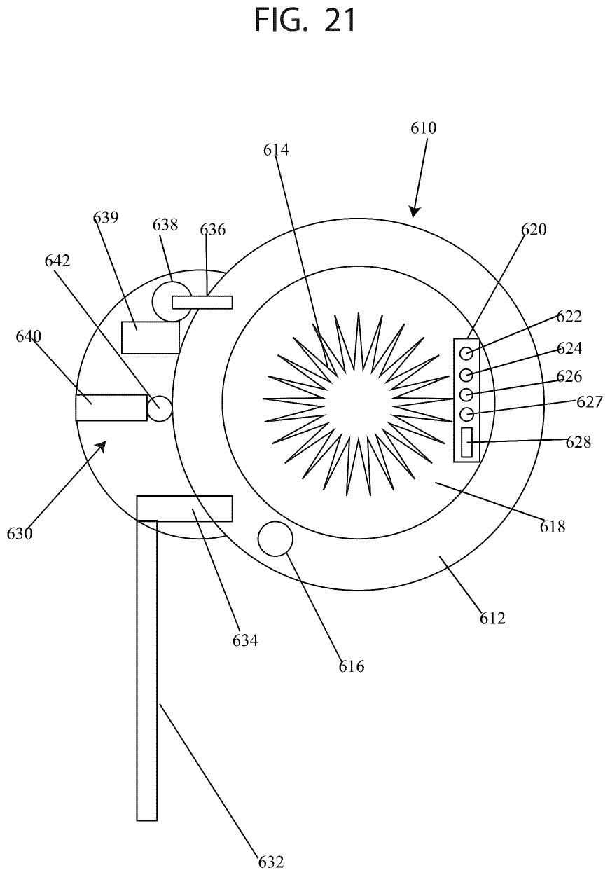

FIG. 21 is a top view of one embodiment of the air purification device 610. The air purification device includes a first housing 612 which can be a cylindrical housing. The first housing 612 includes fan openings 614. There is a fluid input into 614 disposed on a top cover 618 of the housing 612. The fluid input into 614 is configured to receive a cartridge of biological material which can be added to biological solution. Biological solution is formed from a combination of biological material being combined with another fluid such as water.

There is a controller board 620 which includes a plurality of buttons either formed as discrete tactile buttons or buttons formed as part of a touch screen. The buttons include buttons 622, 624, and 626 as well as a display screen 628. Display screen 628 can either be a full display screen or a display screen in combination with additional optional buttons. At least one button such as button 622 can be an on/off button. An addition button 624 can be a fan increase button, while another button 626 can be for example a fan decrease button. There is also another button 627 which can be a mode button so that a user can change the mode so that buttons 624 and 626 are for raising and lowering the preferred level of fluid such as biological solution formed from a combined biological material and water. Screen 628 indicates whether there is a condition that needs to be addressed, or warning sign such as a call for more water, and also indicates the number of RPM that the fan is spinning.

There is also external housing 630. This external housing is configured to house a water input or fluid input 634 which is configured to receive water from a water inlet line. The water input or fluid input 634 can be in the form of a solenoid which is in communication with controller 620 (See also controller 700 in FIG. 3). There is also a water or biological solution outflow line which flows fluid into external or second housing 630. External or second housing 630 is coupled to main housing 612 so that it forms a watertight seal. Both the water input and the water outflow lines are fluidly in connection with the housing 12 and allow water to flow in through the fluid input 634 or biological solution to flow out through fluid outflow line 636. There is a water level sensor 639 which sits in second housing 630 to monitor the water level in this second housing 630.

Another water level sensor 640 has a lens 642. This water level sensor 640 can be in the form of any suitable water level sensor but in this embodiment is an infrared water level sensor which shines an infrared light across the internal expanse of housing 612 to determine the level of the biological solution inside of housing 612. Lens 642 can be in the form of any suitable lens in any suitable form, and which is formed as a prism which can be configured to divide or expand a fluid level beam extending across the inside of housing 612 to determine the level of the biological solution inside of housing 612.

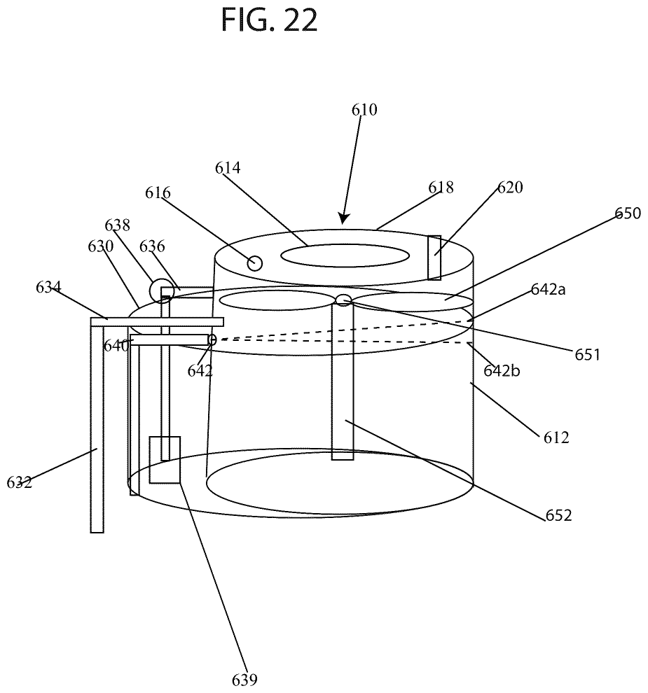

FIG. 22 is a side view of the air purification device. This air purification device as shown includes housing 612, air or fan opening 614, fluid input 616, top 618 as well as controller 620. There is shown a fluid input 634 having a water line 632 coupled thereto a solenoid/pump 638, as well as a fluid outflow 636. A water level sensor 640 includes a light such as a laser light that shines through lens 642 to set the water levels 642a and 642b forming a lower water level 642b and an upper water level 642a.

As shown, there is fan 650 having a motor 651 and a tornado mixer 652 coupled thereto. Motor 651 is configured to drive fan 650 and tornado mixer 652. Fan 650 is configured to draw air into housing 630 and to mix air inside of the housing with the biological solution that is moving inside of the housing due to the tornado mixer 652.

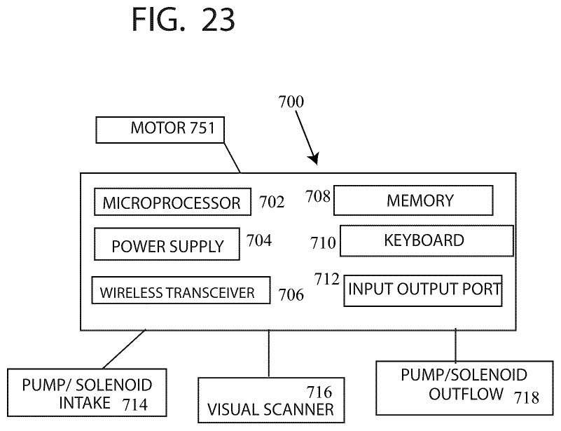

FIG. 23 is a block diagram layout of the air purification device. The air purification device includes a controller 700 which includes a microprocessor 702, a power supply 704, a wireless transceiver 706, and a memory 708. There is also a keyboard 710, and an input/output port 712. Coupled to the controller 700 is an intake pump or solenoid 114. The intake controller 714 is controlled by controller 700 including microprocessor 702. In addition, there is shown visual scanner 716 which is in communication with controller 700. There is also an outflow controller 718 which can be in the form of a one-way check valve. The pump or solenoid is controlled by controller 700, and in particular controlled by microprocessor 702.

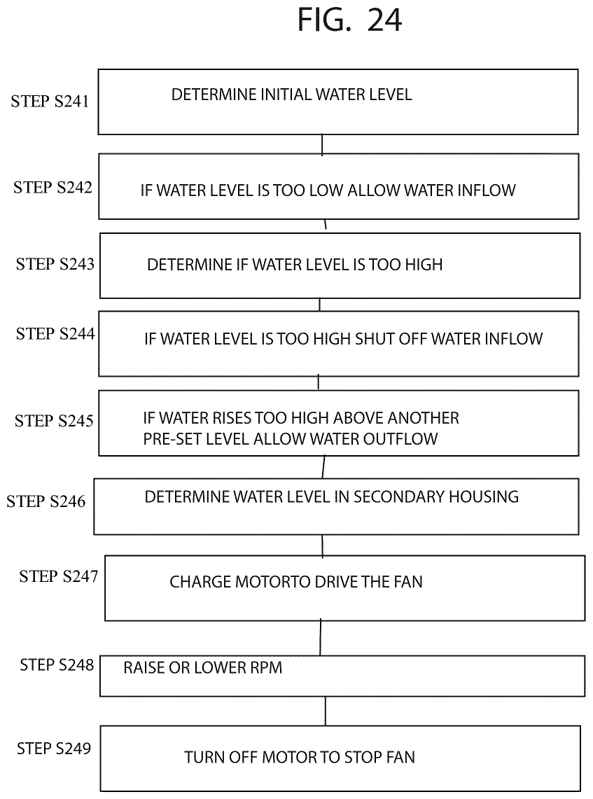

In addition, as shown in FIG. 24 there is a process for controlling the air purification system. The process is controlled by controller 700 which includes microprocessor 702 taking instructions from memory 708. The process starts in step S241 wherein the system determines the water level using visual scanner 716. Visual scanner/Level sensor 716 sends signals to controller 700 so that this information is then processed with microprocessor 702 and stored in memory 708. This information is then received through input/output port 712 or wireless transceiver 706. Step S242 includes determining if the water level is too low or if the water level is too high. This determination occurs in controller 700, and in particular microprocessor 702. Microprocessor 702 reads the current level of the fluid in the first housing and then determines if the water level is too low to allow water level inflow through intake 714 or if in step S243 if the water level is too high then the controller would in step S244 shut off water inflow. Next, in step S245 if the water is determined to be even too high in step S245, above another pre-set level the water can flow out of housing 612 through pump or solenoid outflow 718 (see pump or solenoid 638 in FIGS. 21 and 22), to then call for water or biological solution to flow out from housing 612 and into housing 630.

Next in step S246 the system can through detector 639 determine the fluid or water level inside of secondary or adjacent housing 630. If the water level as detected by detector 639 is relayed to the controller as too high, then the system can shut down.

Next, in step S247 the system can determine whether the fan such as fan 650 should be run. The controller can also control whether the motor speed should be raised or lowered in step S248. Or in step S249 the system (controller) can determine whether to stop the motor such as motor 651 in FIG. 2 (see also motor 751 in FIG. 3).

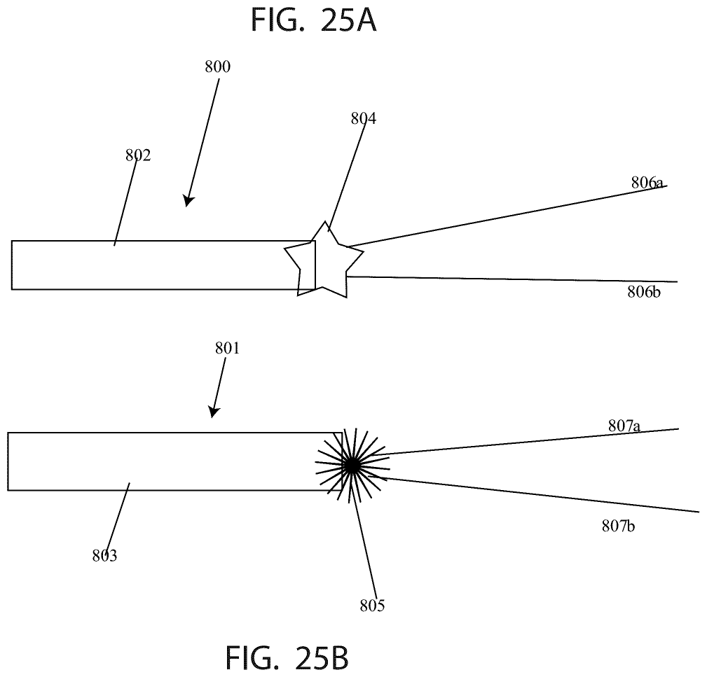

FIG. 25A is a view of a first embodiment of a level sensor 800 which is similar to or the same as water level sensor 640 in FIGS. 21 and 22 or water level sensor or visual scanner 116 in FIG. 23. This sensor 800 includes a lens 804 configured to work with light source 202 to create an upper-level line 806a and a lower-level line 806b to set the upper and lower biological solution levels inside of housing 630.

FIG. 25B is a view of the second embodiment of level sensor 801. In this view there is a light source 803 which shines through a lens 805 and which is configured to create an upper-level line 807a, and a lower-level line 807b for setting the upper and lower levels of the biological solution inside of housing 630. Accordingly, the readings by the controller of the upper-level line 806a or 807a are to determine whether the controller should shut off water inflow, while the lower-level lines 806b and 807b are for whether to call for additional water into housing 630.

Accordingly, while at least one embodiment of the present invention have been shown and described, it is to be understood that many changes and modifications may be made thereunto without departing from the spirit and scope of the invention as defined in the appended claims.

What is claimed is:

1. An air purification system comprising:
a housing;
at least one fan coupled to said housing;
at least one controller coupled to said housing;
at least one air intake formed in said housing;
at least one biological solution comprising a biological agent and water, said biological solution disposed in said housing;
at least one spray nozzle disposed in said housing;
at least one pump configured to pump said biological solution through said at least one spray nozzle to spray said biological solution from said spray nozzle;
at least one screen positioned in said housing, said at least one screen configured to interact with air flowing through the housing, wherein said at least one screen is oriented in a substantially vertical manner;
at least one controller configured to control said pump and said at least one fan.

2. The air purification system as in claim 1, wherein said at least one controller comprises a microprocessor.

3. The air purification system as in claim 1, wherein said at least one controller controls a flow rate of said pump.

4. The air purification system as in claim 1, wherein said at least one controller controls an rpm of said fan.

5. The air purification system as in claim 1, wherein said at least one housing is a substantially rectangular housing with an air inlet on at least one side of the housing and an air outflow on an opposite side of said housing.

6. The air purification system as in claim 1, wherein said at least one screen comprises a plurality of screens extending substantially vertically when the housing is placed on a substantially horizontal surface wherein at least one of said plurality of screens has at least one opening allowing for fluid flow inside of the housing.

7. The air purification system as in claim 6, wherein said plurality of screens comprises at least two screens.

8. The air purification system as in claim 7, wherein said at least two screens comprise a first screen and a second screen wherein said first screen has a first end and a second end and said second screen has a first end and a second end wherein said first end of said first screen is positioned closer to said first end of said second screen than said second end of said first screen is spaced away from said second end of said second screen.

9. An air purification system comprising:
a main housing;
a side housing;
a fan coupled to the main housing;
at least one controller coupled to the housing;
at least one biological solution disposed in the housing;
at least one pump configured to pump fluid into the housing;
at least one electric motor configured to drive the fan;
at least one visual level sensor configured to determine a fluid level in said main housing;
at least one valve to control water intake;
wherein said at least one controller is in communication with said at least one visual level sensor, said at least one pump, said at least one fan, and said at least one solenoid-valve to control an amount of fluid pumped into the main housing, control a rotation rate for said fan.

10. The air purification system as in claim 9, wherein the visual level sensor is an infrared sensor.

11. The air purification system as in claim 9, further comprising at least one overflow system, said overflow system being configured to allow fluid to flow from said main housing into said side housing.

12. The air purification system as in claim 9, wherein said overflow system further comprises at least one one-way valve.

13. The air purification system as in claim 9, wherein said overflow system further comprises at least one one-way valve.

14. The air purification system as in claim 9, wherein said side housing is coupled to said main housing.

15. The air purification system as in claim 9, wherein said main housing has a substantially cylindrical cross section.

16. The air purification system as in claim 15, wherein said side housing has a substantially partially cylindrical cross-section.
